(19) United States
(12) Patent Application Publication
(10) Pub. No.: US 2007/0184309 A1
(43) Pub. Date: Aug. 9, 2007

(54) Methods for use of a photobiofuel cell in production of hydrogen and other materials

(76) Inventors: John Devens Gust Jr, Mesa, AZ (US); Ana L. Moore, Scottsdale, AZ (US); Thomas A. Moore, Scottsdale, AZ (US); Alicia Brune, Tempe, AZ (US)

Correspondence Address: NEEDLE & ROSENBERG, P.C., SUITE 1000, 999 PEACHTREE STREET, ATLANTA, GA, 30309-3915, US

(21) Appl. No.: 10/558,598

(22) PCT Filed: Jun. 1, 2004
(86) PCT No.: PCT/US04/17463

§ 371 Date: Feb. 23, 2007

Related U.S. Application Data
(60) Provisional application No. 60/474,332, filed on May 30, 2003.
(60) Provisional application No. 60/475,066, filed on May 30, 2003.

(51) Int. Cl. H01M 8/16; C12P 3/00

(52) U.S. Cl. 429/2; 435/168

(57) ABSTRACT

The invention provides methods for the in situ production of hydrogen and for the synthesis of high value/energy chemical products from low value/energy organic material.

### BACKGROUND OF THE INVENTION

During photosynthesis, plants convert light energy into electrochemical energy, and eventually into chemical potential energy stored in carbohydrates and other compounds. The carbohydrates are oxidized as needed to provide energy to the organism. A new approach to mimicry of the photosynthetic process that involves a dye-sensitized nanoparticulate semiconductor photoanode working in combination with an enzyme-catalyzed biofuel cell is described in Gust et al., “Enzyme-based Photoelectrical Cell for Electric Current Generation” (WO 03/079480). This system achieves simple and direct coupling of the two complementary processes, combines some of the advantages of each approach in a single unit, and can in principle provide more power than either process working independently.

The present inventors have now shown that this system can be used for the in situ production of hydrogen and for the synthesis of high value/energy chemical products from low value/energy organic material.

### SUMMARY OF THE INVENTION

In one embodiment, the present invention relates to a method for producing hydrogen, comprising the steps of providing a photobiofuel cell comprising an electrochemical half-cell comprising a dye-sensitized photoanode operating in an aqueous medium, said medium comprising NADH, a fuel, and an enzyme selected to provide reducing equivalents to maintain NADH levels; an electrode, the electrode electrically coupled to a catalyst and connected to the photoanode by an electrical conductor; and a light source; and illuminating the photoanode with light to thereby produce hydrogen.

In another embodiment, the present invention relates to a method for producing hydrogen, comprising the steps of providing a photobiofuel cell comprising an electrochemical half-cell comprising a dye-sensitized photoanode operating in an aqueous medium, said medium comprising NADPH, a fuel, and an enzyme selected to provide reducing equivalents to maintain NADPH levels; an electrode, the electrode electrically coupled to a catalyst and connected to the photoanode by an electrical conductor; and a light source; and illuminating the photoanode with light to thereby produce hydrogen.

In another embodiment, the present invention relates to a method for converting low energy organic material to high energy material, comprising the steps of providing an electrochemical fuel cell comprising an electrochemical half-cell comprising a dye-sensitized nanoparticulate photoanode operating in an aqueous medium, said medium comprising NADH, a low energy fuel material, and an enzyme selected to provide reducing equivalents to maintain NADH levels; a compartment comprising an electrode, an NADP-dependent hydrogenase, a catalyst and an NADP-dependent oxido-reductase enzyme, the electrode electrically coupled to the catalyst and connected to the photoanode by an electrical conductor, wherein the compartment is coupled to the electrochemical half cell by a semi-permeable device; and a light source; and illuminating the photoanode with light to thereby convert the low energy fuel material to high energy fuel material.

In still another embodiment, the present invention relates to a method for converting low energy organic material to high energy material, comprising the steps of providing an electrochemical fuel cell comprising an electrochemical half-cell comprising a dye-sensitized nanoparticulate photoanode operating in an aqueous medium, said medium comprising NADPH, a low energy fuel material, and an enzyme selected to provide reducing equivalents to maintain NADPH levels; a compartment comprising an electrode, an NADP-dependent hydrogenase, a catalyst and an NADP-dependent oxido-reductase enzyme, the electrode electrically coupled to the catalyst and connected to the photoanode by an electrical conductor, wherein the compartment is coupled to the electrochemical half cell by a semi-permeable device; and a light source; and illuminating the photoanode with light to thereby convert the low energy fuel material to high energy fuel material.

### BRIEF DESCRIPTION OF THE DRAWINGS

By way of example and to make the description more clear, reference is made to the accompanying drawings in which:

FIG. 1 is a schematic diagram illustrating the procedure of photoelectrochemical oxidation of a carbon-containing compound (fuel) by a photosensitizer (S) in the presence of an enzyme and an oxidation-reduction mediator (R).

FIG. 2 is a diagram illustrating the structure of a power-generating cell used in the evaluation of the photoelectrochemical properties and battery properties, wherein the reference numeral 21 indicates a power-generating cell, the reference numeral 22 indicates a silicon plug, the reference numeral 23 indicates a negative electrode, the reference numeral 24 indicates a counter electrode, the reference numeral 25 indicates a reference electrode, the reference numeral 26 indicates an electrolyte, and the reference numeral 27 indicates an air electrode.

FIG. 3 is a diagram illustrating the current-voltage characteristics of the power-generating cell.

FIG. 4 is a diagram illustrating the change of NADH concentration with time a solution containing various combinations of enzymes and methanol.

FIG. 5 is a diagram illustrating the relationship between the amount of NADH consumed in the electrolyte in the power-generating cell and the amount of electrons removed from the power-generating cell by the external circuit.

FIG. 6 is a diagram illustrating the relationship between the consumed amount of NADPH in the electrolyte in the power-generating cell and the amount of electrons removed from the power-generating cell by the external circuit.

FIG. 7A is a diagram illustrating the change of NADH concentration with time in the electrolyte in the power-generating cell; FIG. 7B is a diagram illustrating the change of NADH concentration with time in the electrolyte in the power-generating cell.

FIG. 8 is a diagram illustrating the relationship between the amount of NADH consumed in the electrolyte in the power-generating cell and the amount of electrons taken out of the power-generating cell by the external circuit.

FIG. 9 is a schematic diagram illustrating one embodiment of the present invention wherein a photobiofuel cell is used with a catalyst to produce hydrogen.

FIG. 10 is a graph showing the amount of hydrogen produced as a function of coulombs and as a function of time in a photobiofuel cell according to practice of the present invention. Photoanode and E-TEK Pt Cathode, 520 nm illumination, NADH as electron source.

FIG. 11A is a graph showing cell current for activated E-TEK Pt vs. Photoanode, λ=520 nm, NADH as electron source; FIG. 11B is a graph showing Absorbance of TPP-COOH on TiO₂/FTO Photoanode, Blank TiO₂/FTO Baseline.

FIG. 12 is a schematic diagram of another embodiment of the present invention wherein a photobiofuel cell is used to covert low value/energy organic material to high value/energy material in a closed system.

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the compositions and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

Overview of the Invention. The present inventors have shown that a photobiofuel cell, described below, initially developed to provide electricity, can be used for the in situ synthesis of hydrogen. By attaching a catalyst that catalyzes the synthesis of hydrogen from protons and electrons to the photoanode of the photobiofuel cell using an electrical conductor or other suitable means, hydrogen is produced using the electrochemical potential of the cell. The hydrogen produced may be thought of as coming from the reforming of the fuel component of the photobiofuel cell. The energy for the hydrogen production comes from both the light absorbed by the photoanode and any associated accessory antenna systems and the chemical energy inherent in the fuel source of the photobiofuel cell. The method described converts a variety of fuel sources including carbon-containing waste materials to a valuable fuel in the form of hydrogen. The fuel sources include, but are not limited to, fats, hydrocarbons, carbohydrates, proteins, and any materials that are oxidized, directly or indirectly, by the photoanode. This process is valuable because a single fuel source such as hydrogen is expected to be the common denominator in future energy economies around the world.

This invention could be used stand alone with the photoanode and the fuel source described below or wired in parallel with an oxygen-reducing cathode in the solar biofuelcell. This is possible because certain hydrogenase enzymes are self-regulating in that, in the presence of oxygen, they are inhibited. In the presence of oxygen, the system converts solar energy to electricity with current flow from the photoanode to the cathode. In the absence of oxygen or at low oxygen concentration, the current flow to the cathode is slow, oxygen inhibition of the hydrogenase enzyme is reduced, and hydrogen synthesis is accelerated.

The hydrogen produced in any manifestations of this photocell would be a form of stored energy and a solid support medium could be employed to increase the energy density.

An additional embodiment of the present invention comprises a method for coupling the in situ synthesis of high value/energy chemical products to the oxidation of low value/energy reactants in a closed system driven by solar energy from a photobiofuel cell, discussed below. This is accomplished by adding an NADP-dependent hydrogenase enzyme to the hydrogen-producing cell so that NADPH is produced from the high energy hydrogen.

The method involves placing the electrode wired to the photoanode and the hydrogen-evolving hydrogenase in a separate compartment with an NADP-linked hydrogenase enzyme. The second compartment is connected to the photoanode compartment by a suitable membrane, frit or salt bridge and the wire or suitable electrical connection between the photoanode and the hydrogen-evolving hydrogenase. The NADPH-linked hydrogenase in the second compartment catalyzes the process NADP⁺+H₂→NADPH+H⁺ which is driven to the right by the supply of H₂ from the hydrogen-evolving hydrogenase wired to the output side of the photoanode. The output side of the photoanode is poised at a highly negative potential by the light-driven processes described below. The hydrogen it produces carries this negative potential as chemical potential in the half reaction H₂→2H⁺+2e⁻.

The NADPH produced from NADP⁺+H₂→NADPH+H⁺ poises the NADP/NADPH redox couple higly reducing for the synthesis of high value/energy chemical compounds. NADPH is the reductant necessary for the bio or biomimetic synthesis of a wide variety of high energy/value reduced carbon compounds useful in myriad processes including, but not limited to, medical, biotechnological, cosmetic, and agricultural processes. The specific high value/energy product obtained is determined by the NADPH-linked oxido-reductase enzyme used for its synthesis. This process is valuable because it makes possible the conversion in a closed system of a low value/energy mixture of compounds, including waste organic materials, to a high value/energy single compound suitable as an energy source for an in situ fuel cell or as a valuable chemical product. The process is driven by solar energy and the chemical energy inherent in the fuel materials. The conversion of the low value/energy fuel material is high because the input side of the photoanode is poised highly oxidizing by the oxidation potential of the photosensitizer radical cation.

### Photobiofuel Cell

A photobiofuel cell (also referred to as a photoelectrochemical cell, a photobiological fuel cell or a photocell) suitable for practice of the present invention as been described in Gust et al., WO 03/079480 (“Enzyme-based Photoelectrochemical Cell for Electric Current Generation”), the entire contents of which are hereby incorporated by reference. The photoelectrochemical cell includes a positive electrode and a negative electrode provided as constituents with an electrolyte interposed between them. When the negative electrode receives electrons from a carbon-containing fuel compound via a photosensitizer compound which produces an oxidant and electrons when irradiated with light, an oxidation-reduction mediator which supplies electrons to the oxidized photosensitizer molecule, and enzymes that catalyze oxidation of the carbon-containing fuel compound, an electromotive force is generated across the positive electrode and the negative electrode. This makes it possible to utilize chemical energy accumulated in the carbon-containing compound directly as electrical energy in a form biased by light radiation energy.

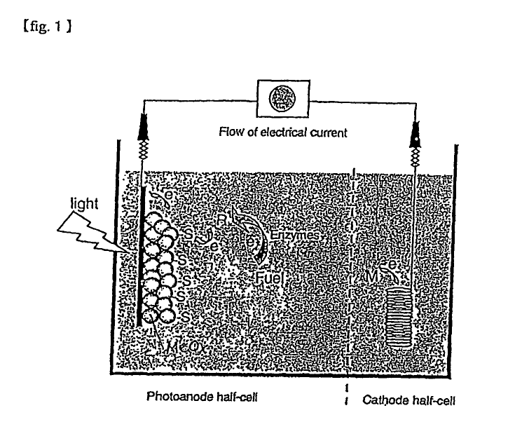

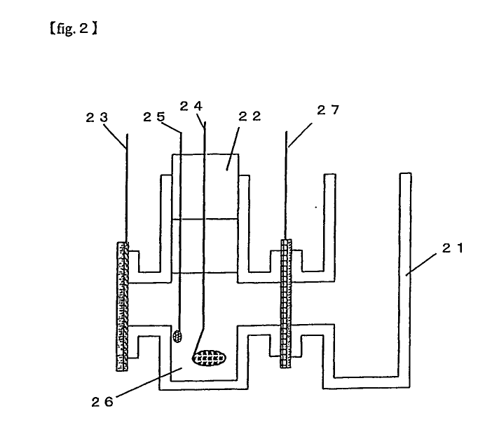

FIG. 1 illustrates a schematic diagram of the configuration of a photobiological fuel. FIG. 1 illustrates how electrons (E−) which have been possessed by a carbon-containing compound (fuel) are released from the carbon-containing compound and ultimately flow through an external electric circuit (load) from the negative electrode to the positive electrode. The electron flow within the cell occurs via a photosensitizer compound (S) retained in or on an oxide semiconductor (MeOx) shown in a spherical form. When irradiated with light, sensitizer S produces an excited state S*. The excited state S* injects an electron into the semiconductor, leaving an oxidant S⁺. The electron (E⁻) has been biased by light irradiation to the optical excitation energy of S (the difference in energy between S and S*). The electron does work in the external circuit, and reaches the positive electrode, where it is used in a reduction reaction with material M. In this manner, an electromotive force is generated across the positive electrode and the negative electrode, generating electricity. During the process, the oxidized photosensitizer S⁺ is reduced to its original form by a redox mediator R, generating R in an oxidized form. The enzyme (s) reduce the oxidized redox mediator back to its original form, obtaining the necessary electrons from oxidation of the carbon-containing fuel compounds. Thus, neither photosensitizer S nor redox mediator R are consumed.

The photosensitizer compound S, which produces an oxidant S⁺ and an electron when irradiated with light, is disposed on an oxide semiconductor. This compound may have a single light absorption peak or a plurality of light absorption peaks in a wavelength range of ˜300 nm to ˜1,000 nm. Such a compound may be a metal complex dye, organic dye, or the like. Examples of a metal complex dye are ruthenium complex dyes or platinum complex dyes having biquinoline, bipyridyl, phenanthroline, or thiocyanic acid or derivatives thereof as ligands. Examples of organic dyes which may also contain metal atoms are porphyrin-based dyes having a single porphyrin ring or a plurality of porphyrin rings. The porphyrin rings may be metal free, or contain zinc (Zn), magnesium (Mg), or the like as a central atom. Examples of such a porphyrin-based dye include those represented by the general formulae P1 to P6 below. Examples of organic dyes are 9-phenylxanthene-based dyes, merocyanine-based dyes, polymethine-based dyes, or the like. In particular, the compound P1, 5-(4-carboxyphenyl)-10,15,20-(4-methylphenyl) porphyrin, having the structure shown below has a high light absorption efficiency and also high affinity for the oxide semiconductor so that it cannot be easily eluted with the electrolyte and thus can be kept stable even after a prolonged contact with the electrolyte. Further, since generation of excited electrons from the compound P1 by irradiation with light can occur over a prolonged lifetime, the compound P1 can exhibit high photoelectric conversion efficiency to advantage. The disposition of a compound which produces an oxidant and an electron when irradiated with light on an oxide semiconductor makes it possible to quickly move an excited electron produced by irradiation with light to the oxide semiconductor and makes less likely the recombination of the oxidant and excited electron produced by irradiation with light, thereby keeping the efficiency of reception of electrons from the carbon-containing compound into the external electrical circuit higher.

As the oxide semiconductor, tin dioxide (SnO₂), titanium dioxide (TiO₂), zinc oxide (ZnO), tungsten oxide (WO₃) or composites thereof such as TiO₂—WO₃ may be used.

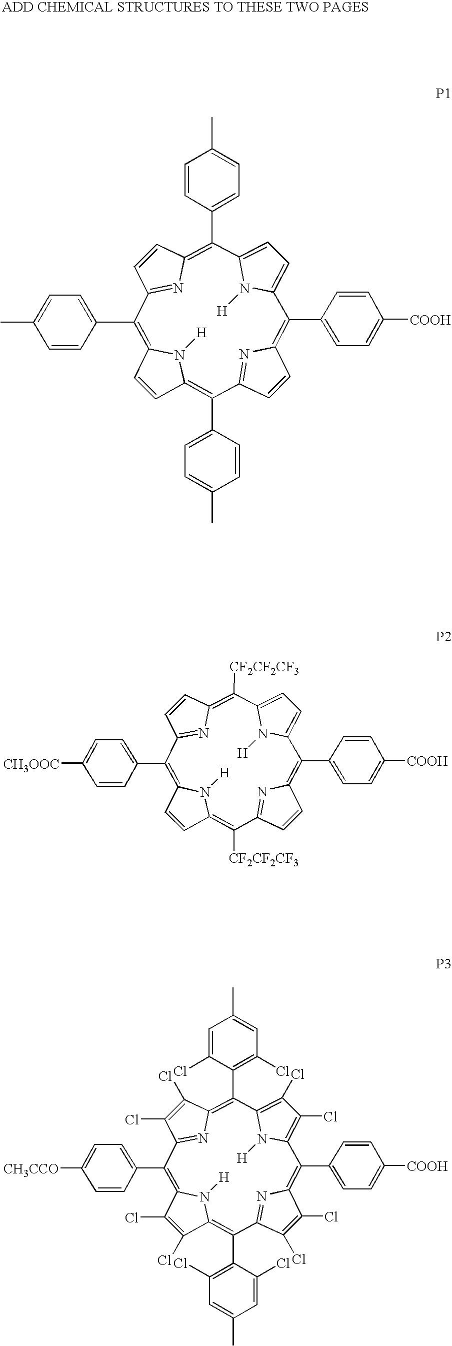

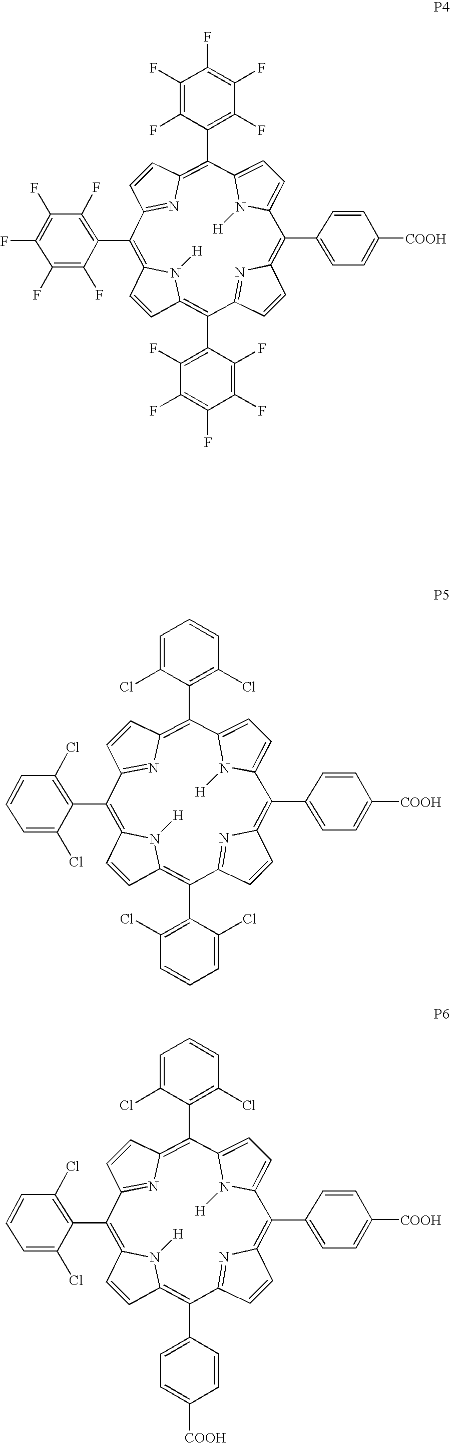

Examples of the carbon-containing fuel compound used in photoelectrical fuel cells, which may be one that is cyclically regenerated in a natural biosphere, include carbohydrates (e. g., sugars, starches), lipids, hydrocarbons, alcohols, aldehydes and organic acids. Such compounds can be produced from carbon dioxide gas and water by photosynthesis and then accumulated. The solar energy stored in these compounds is used as chemical energy via the metabolism of a organism, producing carbon dioxide gas. This forms a clean cyclic system.

Examples of the oxidation-reduction mediator (R) which receives electrons from the carbon-containing fuel compound through the mediation of enzymes and supplies electrons to the oxidant (S⁺) produced by irradiation with light to regenerate the original photosensitizer compound (S) are a quinone/hydroquinone oxidation-reduction couple, the NAD⁺/NADH oxidation-reduction couple, the NADP⁺/NADPH oxidation-reduction couple, the I₂/I₃⁻ oxidation-reduction couple, and metal proteins having an oxidation-reduction capacity such as ferredoxin and myoglobin.

The enzymes, which catalyze the transfer of electrons from the carbon-containing fuel compound to an oxidized form of the oxidation-reduction mediator R, are not specifically limited. In practice, however, dehydrogenase enzymes may be used singly or in combination depending on the kind of the carbon-containing fuel compound. In the case where the fuel is glucose, an enzyme system containing at least glucose dehydrogenase (GDH) may be used.

In the case where the fuel is D-glucose-6-phosphate, an enzyme system containing at least D-glucose-6-phosphate dehydrogenase (G-6-PDH) or at least G-6-PDH and 6-phosphogluconate dehydrogenase (6-PGDH) may be used.

In the case where the fuel is methyl alcohol, an enzyme system containing at least an alcohol dehydrogenase (ADH), an enzyme system containing at least ADH and an aldehyde dehydrogenase (ALDH), or an enzyme system containing at least ADH, ALDH and a formate dehydrogenase (FDH) may be used.

In the case where the fuel is ethyl alcohol, an enzyme system containing at least an alcohol dehydrogenase (ADH) or an enzyme system containing at least ADH and an aldehyde dehydrogenase (ALDH) may be used. In the case where a plurality of fuels is used, enzymes corresponding to these fuels may be used in admixture.

As the electrolyte to be incorporated into the photobiological fuel cell, there may be used any material regardless of whether it is an organic material, inorganic material, liquid or solid so far as it allows the movement of anions and/or cations from the positive electrode to the negative electrode and/or from the negative electrode to the positive electrode to cause continuous progress of oxidation-reduction reactions at the positive electrode and the negative electrode. An aqueous solution obtained by dissolving a salt such as KCl, NaCl, MgCl₂, NH₄Cl and Na₂HPO₄, an alkali such as NH₄OH, KOH and NaOH or an acid such as H₃PO₄ and H₂SO₄ in water is safe, causes no environmental pollution, and can be easily handled to advantage. Alternatively, a solution of a quaternary ammonium salt such as pyridinium iodide, a lithium salt such as lithium iodide, an imidazolium salt such as imidazolinium iodide, t-butylpyridine or the like in acetonitrile, methoxyacetonitrile or methoxypropionitrile, an ion exchange membrane made of a polymer material such as fluororesin having sulfonic acid groups, amide groups, ammonium groups, pyridinium groups or the like or a polymer electrolyte such as solution of a salt such as LiBF₄, LiClO₄ and (C₄H₉)₄NBF₄ in a polypropylene oxide, polyethylene oxide, acrylonitrile, polyvinylidene fluoride, polyvinyl alcohol or the like may be used.

The reaction at the positive electrode in the photobiological fuel cell of the invention involves a reduction reaction occurring at a higher (or more anodic) potential than that of the electron taken out of the carbon-containing compound via an optically excited active species (S*) of molecule at the negative electrode. Any reduction reaction can be employed so far as the electron thus taken out is electrochemically received by the positive electrode via the external load.

Examples of the reaction at the positive electrode include reduction reactions of water or oxygen, reduction reactions of hydroxide or oxides such as NiOOH, MnOOH, Pb(OH)₂, PbO, MnO₂, Ag₂O, LiCoO₂, LiMn₂O₄ and LINiO₂, reduction reactions of sulfides such as TiS₂, MoS₂, FeS and Ag₂S, reduction reactions of metal halides such as AgI, PbI₂ and CuCl₂, reduction reactions of halogen such as Br₂ and I₂, reduction reactions of organic sulfur compounds such as quinone and organic disulfide compounds, and reduction reactions of electrically-conductive polymers such as polyaniline and polythiophene.

In particular, the positive electrode is preferably an oxygen electrode for reducing oxygen. In this arrangement, a gas containing oxygen can be used as the positive active material, eliminating the necessity of retaining a positive active material in the battery and hence making it possible to form a battery having a higher energy density.

Any material capable of reducing oxygen may be used as the oxygen electrode. Examples of such an oxygen-reducing material include activated charcoal, manganese oxide including MnO₂, Mn₃O₄, Mn₂O₃ and Mn₅O₈, platinum, palladium, iridium oxide, platinum-ammine complexes, cobalt-phenylenediamine complexes, metal porphyrins (metal: cobalt, manganese, zinc, magnesium, etc.), and perovskite oxides such as La(Ca)CoO₃ and La(Sr)MnO₃.

Example 1 of a Photoelectrical Chemical Cell (from Gust et al., WO 03/079480)

As a photosensitizer compound which produces an oxidant and an electron when irradiated with light, 5-(4-carboxyphenyl)-10,15,20-(4-methylphenyl) porphyrin (P1) was used as a typical representative of a porphyrin photosensitizer used to prepare a negative electrode.

Preparation Of The Negative Electrode. A light-transmitting glass substrate with a thickness of 1 mm bearing a thin film of electrically conducting indium-tin oxide (ITO) with a surface resistivity of 10-12 Ω/cm² was used to prepare the negative electrode. A 1% by weight aqueous dispersion of particulate tin dioxide (SnO₂) having an average particle diameter of 10 nm was deposited on the ITO film by spraying or otherwise applying layers over a hot plate. The electrode was dried at a temperature of 80° C., and then sintered at a temperature of 400° C. in air for 1 hour to form a film of particulate SnO₂. Subsequently, the electrode was dipped into a 1-5 mM solution of photosensitizer P1 (dissolved in dichloromethane, toluene, or hexanes) for typically 1 hour, withdrawn from the solution, washed with clean solvent, and dried with a stream of nitrogen gas. The presence of P1 on the electrode particulate surface was confirmed by its absorption spectrum.

Assembly Of Test Cell. The negative electrode thus prepared was then used to assemble a power-generating cell 21 having the structure shown in FIG. 2.

In the power-generating cell 21, the film of particulate SnO₂ on the negative electrode 23 on which the dye P1 is deposited comes in contact with an electrolyte 26. In the electrolyte 26 are disposed a counter electrode 24 which forms a battery in combination with the negative electrode 23 and a reference electrode 25 which gives a reference potential on the basis of which the potential of the negative electrode 23 is measured. Further disposed is an air electrode 27, which forms a battery in combination with the negative electrode 23 instead of the counter electrode 24. The air electrode 27 was prepared by embedding a mixture of Mn₂O₃ powder, activated charcoal powder, acetylene black powder and polytetrafluoroethylene (PTFE) binder on a nickel screen having a thickness of 0.2 mm. The reference numeral 22 indicates a silicon plug for fixing the counter electrode 24 and the reference electrode 25 to the power-generating cell 21.

Operating Characteristics Of Photoelectric Power-Generating Cell. A power-generating cell (a) was assembled as described above, using the negative electrode 23, a platinum (Pt) counter electrode (24), and an electrolyte 26, which is a 0.1 M aqueous solution of sodium acetate (NaOAc) containing 2.5 mM hydroquinone (QH₂) as an oxidation-reduction mediator (R).

A power-generating cell (b) was assembled as described above, using the negative electrode 23, a platinum (Pt) counter electrode 24 immersed in a saturated aqueous solution of potassium sulfate free of dissolved oxygen and isolated from the electrolyte 26 by an ion-permeable membrane, and electrolyte 26, which is a 0.1 M aqueous solution of sodium acetate (NaOAc) containing 2.5 mM nicotinamide-adenine dinucleotide in the reduced form (NADH) as an oxidation-reduction mediator (R).

A power-generating cell (c) was assembled as described above, using the negative electrode 23, a counter electrode 24, which is a mercury/mercury (I) sulfate electrode separated from electrolyte 26 by an ion permeable membrane, and electrolyte 26, which is an 0.1 M aqueous solution of sodium acetate containing 2.5 mM nicotinamide-adenine dinucleotide in the reduced form (NADH) as an oxidation-reduction mediator (R).

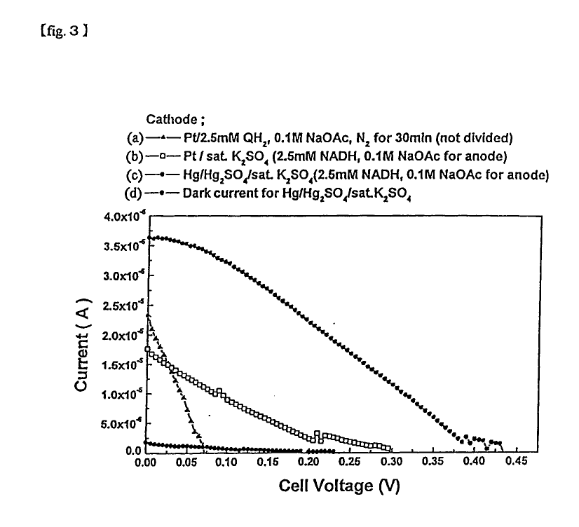

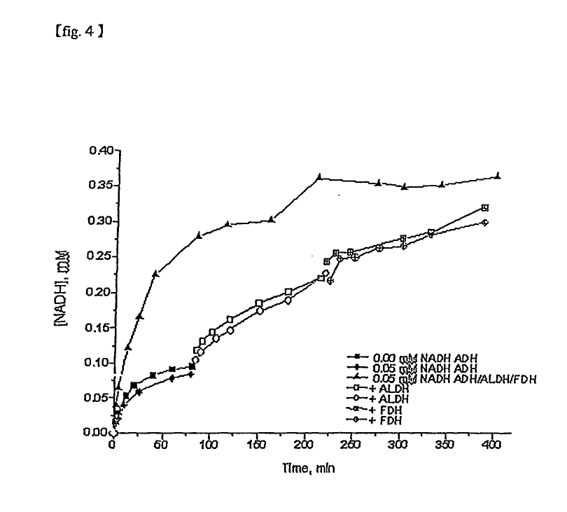

FIG. 3 illustrates current-voltage characteristics of these power-generating cells developed when they are irradiated with light having a wavelength of 520 nm. In FIG. 3, the curves (a), (b) and (c) indicate the current-voltage characteristics of the power-generating cells (a), (b) and (c), respectively. All the power-generating cells (a), (b) and (c) work as batteries, although they show differences in current-voltage characteristics. In FIG. 3, the curve (d) indicates the current-voltage characteristics of the power-generating cell (a) developed when it is not irradiated with light. When not irradiated with light, a power-generating cell gives little or no output current.

In these cells, the dye deposited on the negative electrode acts as a photosensitizer compound (S) which upon irradiation produces an excited state (S*). In contact with the metal oxide, it injects an electron into the oxide particle, producing an oxidant (S⁺). The external circuit removes the electron thus produced, where it is then measured as output current of the battery. In cells of type (c), the oxidant (S⁺) receives an electron from the oxidation-reduction mediator NADH (or in some cases QH₂), regenerating S. Thus, in a cell lacking enzymes or carbon-containing fuel compounds, the supply of electrons to the external circuit lasts until NADH (or QH₂ when that is used as the oxidation-reduction mediator) is consumed.

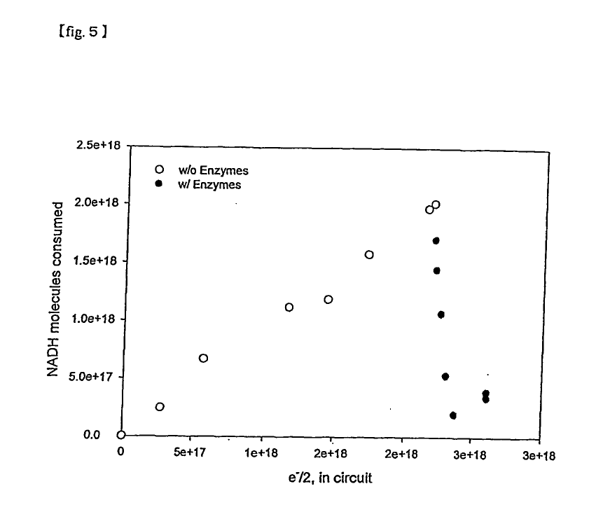

An assay (not performed in the cell) was done to test the production of NADH when NAD⁺ is present and methanol is used as the carbon-containing fuel compound. An aqueous solution of pH 8.0 containing 1 M NaCl, 5 mM oxidized nicotinamide-adenine-dinucleotide (NAH⁺) and methanol with 0.0 and 0.05 mM reduced nicotinamide-adenine-dinucleotide (NADH) and an alcohol dehydrogenase (ADH), an aldehyde dehydrogenase (ALDH) and a formate dehydrogenase (FDH) as enzymes added thereto. The change of NADH concentration with time during irradiation with light is shown in FIG. 4.

In FIG. 4, the symbol ▴ indicates the change of NADH concentration in a solution having 5.0 mM NAH⁺, 0.05 mM NADH, ADH, ALDH and FDH added thereto. The symbol ● indicates the change of NADH concentration in a solution having 5 mM NAH⁺, 0.05 mM NADH and ADH added thereto. The symbol ∘ indicates the change of NADH concentration in the same solution as in (●) but having ALDH added thereto after the lapse of a predetermined time from the addition of NADH and ADH. The symbol ∘+ indicates the change of NADH concentration in the solution (∘) but having FHD added thereto after the lapse of a predetermined time from the addition of NADH, ADH and ALDH. The symbol ▪ indicates the change of NADH concentration in the electrolyte having 5 mM NAH⁺ and ADH added thereto. The symbol □ indicates the change of NADH in the same solution as the solution ▪ but having ALDH added thereto. The symbol □⁺ indicates the change of NADH in the same solution as the electrolyte □ but having FDH added thereto. All these tests were observed to have an increase of NADH concentration, demonstrating that an electron moves from methanol to NAH⁺ through the mediation of the enzyme to produce NADH. In other words, as shown in FIG. 4, NADH is formed from NAH⁺ through the mediation of enzymes that utilize methanol. These results imply that as long as methanol is present in a power-generation cell with the mediation of enzymes, NADH used by the negative electrode will be regenerated, and power generation can be maintained under irradiation with light.

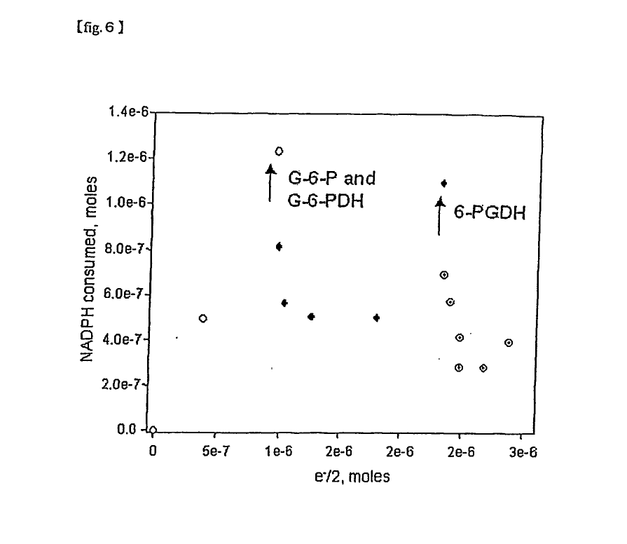

FIG. 5 is a graph illustrating the relationship between the amount of electrons taken out of the cell by the external circuit (abscissa) and the amount of NADH consumed by irradiation with light (ordinate) in a power-generating cell of type (c), with the electrolyte 26 containing NADH and methanol. The symbol ∘ indicates the relationship between the amount of NADH consumed and the amount of electrons produced in the external circuit when the electrolyte is free of enzymes. This relationship shows that the consumed amount of NADH and the amount of electrons are proportional to each other, demonstrating that electrons released from NADH are properly taken out by the external circuit. The symbol indicates the relationship between the amount of NADH consumed and the amount of electrons produced in the external circuit when ADH, AIDH and FDH are added to the electrolyte as the enzyme system. Under these conditions, little or no NADH is consumed, regardless of the amount of electrons removed by the external circuit. In other words, NADH releases electrons to form NAH⁺, which then receives electrons from methanol through the mediation of the enzymes thus added to regenerate NADH. This state of little or no NADH consumption lasts as long as methanol is present in the electrolyte. In other words, power generation continues while methanol is present.

In these and other experiments, the concentration of NADH in the electrolyte was determined by the intensity of the peak present in the vicinity of 340 nm in the UV absorption spectrum of NADH.

In the present example, tin oxide (SnO₂) was used as the oxide semiconductor. The same evaluation was made with particulate TiO₂, and could be made with films of particulate metal oxide such as ZnO and TiO₂—WO₃ instead of SnO₂. The same evaluation using SnO₂was also made on the compounds P2, P3, P4, P5 and P6 instead of the compound P1 as a photosensitizer. These compounds also produce an oxidant and an electron when irradiated with light. As a result, these power-generating cells will exhibit operating characteristics similar to that of P1.

### Example 2 of a Photoelectrical Chemical Cell (from Gust et. al., WO 03/079480)

A power-generating cell was formed by the same type of negative electrode 23 as used in Example 1, platinum (Pt) as counter electrode 24 and an aqueous buffered solution at pH 8.0 containing NADP⁺/NADPH as the oxidation-reduction mediator in the electrolyte. D-Glucose-6-phosphate (G-6-P) was used as a carbon-containing fuel compound. D-glucose-6-phosphate dehydrogenase (G-6-PDH) and 6-phosphogluconate dehydrogenase (6-PGDH) were used as an enzyme system.

FIG. 6 is a graph illustrating the relationship between the amount of electrons removed by the external circuit from the electrolyte (abscissa) and the amount of NADPH consumed during irradiation with light (ordinate). The symbol ∘ indicates the relationship between the amount of NADPH consumed and the amount of electrons injected into the external circuit with an the electrolyte free of fuel and enzyme. This relationship shows that the amount of NADPH consumed and the amount of electrons produced are proportional to one another, demonstrating that electrons released from NADPH are removed by the external circuit. The symbol ● indicates the relationship between the amount of NADPH consumed and the amount of electrons removed in the electrolyte to which has been added G-6-P as a carbon-containing fuel compound, and the enzyme G-6-PDH. The amount of NADPH consumed is greatly reduced. This result shows that NADPH has released electrons to form NADP⁺, which then receives electrons from G-6-P to regenerate NADPH through the mediation of the added enzymes. The amount of NADPH consumed remains approximately constant regardless of the number of electrons removed by the external circuit. This state lasts as long as G-6-P is present in the electrolyte. At later times, when G-6-P is entirely oxidized to gluconolactone-6-phosphate, the amount of NADPH consumed again rises. The gluconolactone-6-phosphate hydrolyzes in the electrolyte to 6-phosphogluconate (6-PG). The amount of NADPH consumed increases in proportion to the amount of electrons removed into the external circuit. When 6-PGDH, which is an enzyme that oxidizes 6-PG, is added to the electrolyte, the amount of NADPH consumed shows a sudden drop as shown by the symbol ∘+ in FIG. 6. Electrons are again supplied by 6-PG, which is a fuel, to regenerate NADPH. The reception of electrons by the external circuit lasts as long as 6-PG is present in the electrolyte.

In this example, the oxide semiconductor was tin oxide (SnO₂). Similar evaluations could be made on films of particulate TiO₂ or other particulate metal oxides such as ZnO and TiO₂—WO₃.

### Example 3 of a Photoelectrical Chemical Cell (from Gust et al., WO 03/079480)

A power-generating cell was formed by the same negative electrode 23 as used in Example 1, platinum (Pt) as a counter electrode 24 and a buffered solution at pH 8.0 containing a 0.5 mM NADH and 10 mM NAH⁺ as an electrolyte. Ethanol (CH₃CH₂OH) was used as a carbon-containing fuel compound. The nicotinamide-adenine-dinucleotide couple (NADH)/(NAH⁺) was used as the oxidation-reduction mediator. An alcohol dehydrogenase (ADH) and an aldehyde dehydrogenase (ALDH) were used as an enzyme system.

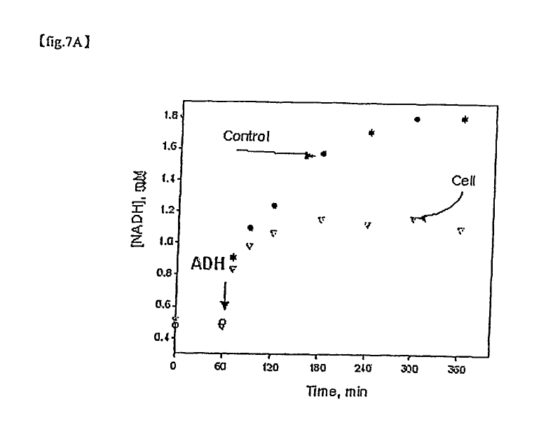

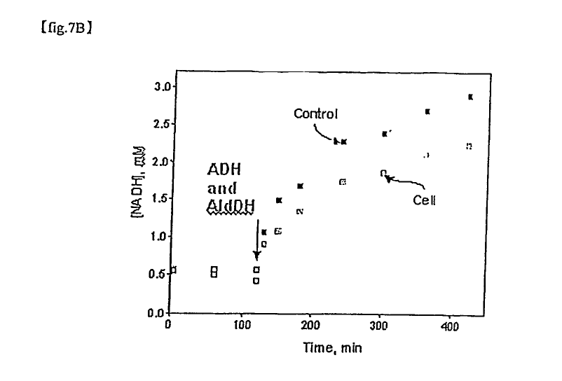

FIG. 7A is a graph illustrating the change of NADH concentration with time in the electrolyte containing ethanol. An alcohol dehydrogenase (ADH) enzyme system was added thereto after 60 minutes. In FIG. 7A, the symbol ▾ indicates the change of NADH concentration in the power-generating cell containing ethanol and ADH. The concentration of NADH increases with light irradiation time until it reaches a value determined by the amount of electrons removed by the external circuit and the amount of ethanol in the electrolyte. On the contrary, the symbol ∘ indicates the change of NADH concentration in an identical electrolyte put in a container free of electrodes, but having ADH added thereto under the same conditions as in the power-generating cell. In FIG. 7A, these data are noted as “Control”. Since the Control has no electrodes, electrons are not removed by the external circuit. The NAH⁺ in the electrolyte receives electrons from ethanol and is converted to NADH through the mediation of ADH. Thus, the concentration of NADH continues to increase with time.

A power-generating cell was formed by a negative electrode 23, an air electrode 27 and an electrolyte at pH 8 containing ethanol, NADH and ADH as an enzyme system. When irradiated with sunlight, the power-generating cell operated as a photobiological fuel cell having a voltage of about 0.65 V.

FIG. 7B is a graph illustrating the change of NADH concentration with time in the electrolyte containing ethanol with later addition of an alcohol dehydrogenase (ADH) and aldehyde dehydrogenase (ALDH) as an enzyme system.

In FIG. 7B, the gray empty squares indicate the NADH concentration in the power-generating cell after addition of ethanol, but prior to addition of ADH and ALDH. The NADH concentration is constant. After addition of ADH and ALDH (gray filled squares), the concentration of NADH increases until it approaches a value determined by the amount of electrons taken out by the external circuit and the amount of ethanol and its oxidation product acetaldehyde in the electrolyte. The black empty squares indicate the change of NADH concentration in an electrolyte with ethanol, put in a container free of electrodes, prior to addition of enzymes. The concentration is constant. After addition of ADH and ALDH under the same conditions as in the power-generating cell, the rise in NADH concentration is shown as black filled squares. In FIG. 7B, these latter plots are identified as “Control”. Since Control has no electrodes, electrons are not removed by the external circuit. NAH⁺ in the electrolyte receives electrons from ethanol and acetaldehyde, and is converted to NADH through the mediation of ADH and ALDH. Thus, the concentration of NADH continues to increase with time.

A power-generating cell was formed by an electrolyte 23, an air electrode 27, and an electrolyte containing NADH, NAH⁺, ethanol, and ADH and ALDH as an enzyme system. When irradiated with sunlight, the power-generating cell operated as a photobiological fuel cell having a voltage of about 0.65 V.

In the present example, tin oxide (SnO₂) was used as the oxide semiconductor. The same evaluation could be made with films of particulate TiO₂, or other particulate metal oxides such as ZnO and TiO₂.WO₃ instead of SnO₂. The same evaluation could also be made on the compounds P2, P3, P4, P5 and P6 instead of the compound P1 as a photosensitizer compound, which produces an oxidant and an electron when irradiated with light.

### Example 4 of a Photoelectrical Chemical Cell (from Gust et al., WO 03/079480)

A power-generating cell was formed by the same negative electrode 23 as used in Example 1, platinum (Pt) as a counter electrode 24 and a buffer solution having pH 7.3 containing an NAH+/NADH oxidation-reduction mediator in the electrolyte. D-glucose was used as a carbon-containing compound was used. D-glucose-dehydrogenase (GDH) was used as an enzyme.

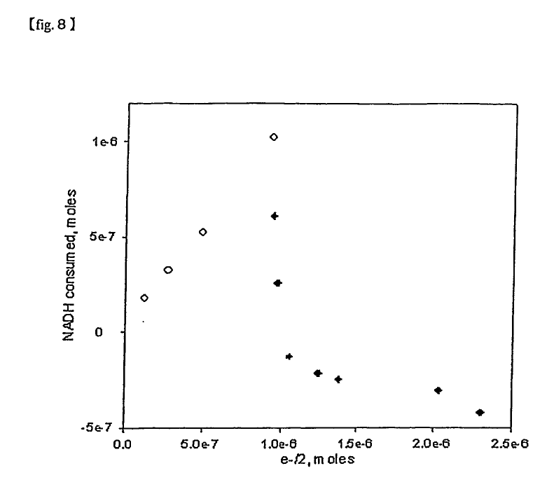

FIG. 8 is a graph illustrating the relationship between the amount of electrons removed by the external circuit from the electrolyte having GDH added thereto as an enzyme system (abscissa) and the amount of NADH consumed during irradiation with light (ordinate). The symbol ∘ indicates the relationship between the amount of NADH consumed and the amount of electrons produced prior to addition of the enzyme, but with D-glucose present. This relationship shows that the amount of NADH consumed and the amount of electrons are proportional to one another, demonstrating that electrons released from NADH are removed by the external circuit. The symbol ∘ indicates the relationship between the amount of NADH consumed and the amount of electrons produced in the external circuit from the electrolyte after addition of the enzyme system.

Before addition of the enzyme, NADH is consumed and oxidized to NAH⁺, with the concurrent production of electrons in the external circuit. After addition of the enzyme, NADH is regenerated and the amount apparently consumed drops slightly below the original amount. In other words, NADH releases electrons to form NAH⁺, which then receives electrons from D-glucose through catalysis by the enzyme to reform NADH. Thus, in the presence of the enzyme, the amount of NADH consumed is kept constant regardless of the number of electrons thus taken out. This state lasts as long as D-glucose is present in the electrolyte.

In the present example, tin oxide (SnO₂) was used as the oxide semiconductor. The same evaluation could be made on films of particulate TiO₂, and on other particulate metal oxides such as ZnO and TiO₂.WO₃, instead of SnO₂. The same evaluation could also be made on the compounds P2, P3, P4, P5 and P6 instead of the compound P1 as a photosensitizer compound which produces an oxidant and an electron when irradiated with light.

### Method for the Synthesis of Hydrogen using a Photobiofuel Cell

This invention consists of a method for the in situ synthesis of hydrogen using electrical current from the output side of the photoanode and the fuel and coenzymes NAD or NADP and associated enzymes that make up the input components of the photoanode discussed above.

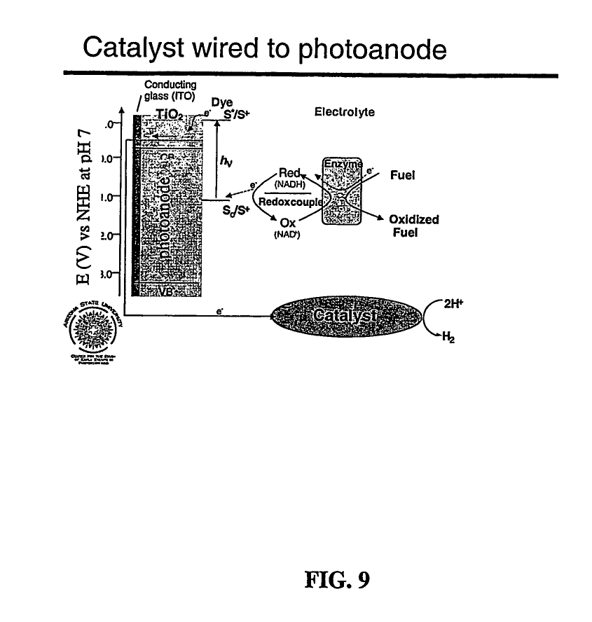

A catalyst that catalyzes the synthesis of hydrogen from protons and electrons is attached to the photoanode employed in the cell previously disclosed by an electrical conductor or other suitable means so that sufficient electrochemical potential is provided to produce hydrogen. See FIG. 9.

The hydrogen produced may be thought of as coming from the reforming of the fuel component of the photobiofuel cell previously disclosed. The energy for the hydrogen production comes from both the light absorbed by the photoanode and any associated accessory antenna systems and the energy inherent in the fuel source of the cell previously disclosed. The method described is useful in that it converts a variety of fuel sources including carbon-containing waste materials and low energy content fuels to a valuable fuel in the form of hydrogen. The fuel sources include, but are not limited to, fats, hydrocarbons, carbohydrates, proteins, and any materials that are oxidized, directly or indirectly, by the photoanode.

In order to operate, a catalyst for hydrogen production must have a source of protons from an aqueous solution at moderate pH and electrons with sufficient reduction potential to form hydrogen (E_{1/2}=0.42 V at pH 7). Hydrogenase enzymes are examples of such catalysts and are found in a large variety of organisms and catalyze the formation of hydrogen from protons and electrons. Other catalysts include various platinum-based materials such as E-TEK Pt/C. Other suitable catalysts will be apparent to those of skill in the art.

The basis of the method is a photoelectrochemical fuel cell. Fuel cells carry out exergonic oxidation and reduction chemical reactions in two half-cells, and use the resulting energy to generate electromotive force and electrical current flowing between an anode and a cathode. In this invention, electron flow from the photoanode is diverted from the cathode to a catalyst, catalyzing the formation of hydrogen from protons and electrons. The device is a hybrid electrochemical fuel cell consisting of:

a. An electrochemical half-cell consisting of a dye-sensitized nanoparticulate photoanode operating in an aqueous medium, which contains the reducing agent NADH or NADPH that donates electrons into the photo-oxidized dye at the photoanode, thereby being itself oxidized. This half cell also contains appropriate reduced carbon or other fuel materials in conjunction with appropriate enzymes to provide reducing equivalents to maintain the NADH or NADPH levels.

b. A second electrode attached electrically to a catalyst for hydrogen production including any of the class of hydrogen producing enzymes known as hydrogenases. The catalyst-bearing electrode is wired to the photoanode by an electrical conductor and may be in the photoanode compartment or coupled to the photoanode half-cell via a semi-permeable device such as a membrane, frit or salt bridge.

c. A suitable light source.

Details of Half-Cell Function

Photoanode. One component of the photoanode (left side of FIG. 9) is a conductive surface, which may be transparent to light. Indium tin oxide coated glass or fused silica or fluorine tin oxide coated glass or fused silica are examples of transparent conductive materials. The surface of this material is covered with a layer of wide band gap semiconductor nanoparticles. Examples of suitable materials are tin dioxide and titanium dioxide. Onto the surface of the nanoparticles is deposited a layer of light-absorbing sensitizer dye (S in FIG. 9). This material absorbs light in spectral regions where the excitation source with which the cell will be used has a significant photon flux. The energies of some of its lowest-lying excited states (singlet, triplet) and its first oxidation potential are such that the singlet or triplet state is energetically capable of injection of an electron into the nanoparticulate surface to generate S⁺. This event leads to formation of the oxidized sensitizer, S⁺, and mobile electrons which migrate through the nanoparticulate layer to the conductive surface, and hence into the external electrical circuit. The sensitizer S bears functionality that allows it to bind to the surface of the nanoparticles in a structural arrangement that renders the excited state of S kinetically competent to inject an electron into the nanoparticle layer with suitable efficiency.

Redox Couple. The redox couple consists of NADH/NAH⁺ or NADPH/NADP+, depending upon which species is active towards the fuel material to be oxidized. A mixture may be used with appropriate enzymes to accommodate a variety of fuel sources. As the cell operates upon illumination of the photoanode, the NADH or NADPH present is converted into NAH⁺ or NADP⁺. In this process, two electrons are removed from NADH or NADPH by the oxidized sensitizer S⁺, regenerating S, and electrons pass through the external circuit. The fuel materials in concert with appropriate enzymes regenerate the NADH or NADPH and become oxidized themselves. Thus, S is recycled as a photocatalyst, and NADH or NADPH are recycled, and the electrons flowing through the circuit are used by the hydrogenase to produce hydrogen from protons.

Electrolyte. The half-cell electrolyte is aqueous in nature, providing an environment in which the photoanode, redox couple, and hydrogenase are capable of functioning as described above. The electrolyte contains any necessary buffers, salts or other substances necessary to ensure stable operation of the cell.

Catalyst. Electrons flowing from the photoanode through an appropriate electrical conduit to the catalyst will have sufficient reducing potential to drive the synthesis of hydrogen from protons and electrons according to the chemical equation: 2H⁺+2e⁻→H₂. Among all catalysts for hydrogen production, the hydrogenase enzymes include, but are not limited to, the NiFe hydrogenases from organisms such as Escherichia coli, Nostoc muscorum, Rhodospirillum rubrum, Rhodobacter capsulatus, Chromatium vinosum and others. Fe-only hydrogenases from organisms such as Clostridiium acetobutylicum and others may also be used. The hydrogenase electrode connection could be as described by Reshad et al., (Biochemistry 38, 8992-8999 (1999)) or by other suitable means involving modified electrodes and molecular-level electrical contact techniques as will be appreciated by the skilled artisan.

Configuration. This invention can be used stand alone as shown in FIG. 9 with the photoanode and the fuel source previously disclosed or wired in parallel with an oxygen reducing cathode in the solar photobiofuel cell described above. This is possible because certain of the hydrogenase enzymes are self-regulating in that, in the presence of oxygen, they are inhibited. Under oxygenated conditions, the device acts as a light-energy to electrical-energy converting device with current flow from the photoanode to the cathode. In the absence of oxygen or when the concentration of oxygen is low, the current flow to the cathode (where oxygen is consumed) is slow, oxygen inhibition of the hydrogenase enzyme is reduced, and hydrogen synthesis is accelerated. Functioning in this mode the hydrogen is an energy storage medium, and the system can incorporate solid state storage media for appropriate energy densities.

In the dark or under heavy current demand, the hydrogen produced and stored would be oxidized by the hydrogenase, and would supply electrons to the anode and thereby replace the photocatalytic production of electrons by the photobiofuel cell. Alternatively, this hydrogen oxidation could be used in addition to illumination of the photoanode and consumption of a fuel, thus augmenting the output of the photobiofuel cell. Operation in this mode would require an oxygen tolerant hydrogenase or a means of excluding oxygen from the hydrogenase enzyme, and in this sense is an alternative to the self-regulated operation discussed above.

### EXAMPLE 1

### Quantum Yield of Hydrogen Production, TPP-COOH Photoanode to ETEK Pt/C Cathode

A) Experimental Setup

1) Two compartment cell separated by Nafion N-112 proton exchange membrane.

2) Initial anode solution: 15 mL: 250 mM Tris-HCL pH 8.

3) Initial cathode solution: 3 mL: 250 mM Tris-HCL pH8.

4) A 7 mm×24 mm piece of LT 140E-W Low Temperature ELAT gas diffusion electrode, microporous layer including Pt electrode on woven web (De Nora North America Inc, E-TEK Division) was inserted into cathode cell.

5) Both solutions were bubbled with Ar for one hour.

B) Platinum Surface Activation

1) Ar was removed from cathode cell. Cathode cell was sealed with Thermogreen LB-2 septa (Supelco).

2) A blank TiO₂/FTO anode (ECN) was inserted in the anode cell. Anode cell was Ar bubbled throughout experiment.

3) Electrodes were connected through Keithley 617 electrometer, measuring current.

4) The TiO₂ anode was illuminated from a Xe arc lamp and shortpass filter (λ<1100 nm) for 20 minutes.

C) Hydrogen Production from TPP-COOH Photoanode

1) The blank TiO₂/FTO anode was removed from the anode cell. 43 mg of NADH was added to anode solution, approximating 4 mM NADH in the anode solution.

2) A TPP-COOH (porphyrin) treated TiO₂/FTO photoanode was inserted into the anode cell for rear illumination.

3) The cathode headspace gas (cathode cell remained sealed) was exchanged for Ar to remove any hydrogen produced during the activation period.

4) The photoanode was illuminated by a 520 nm monochromatic light source (1.98 mW/cm²) for 140 minutes. The current being passed was measured throughout.

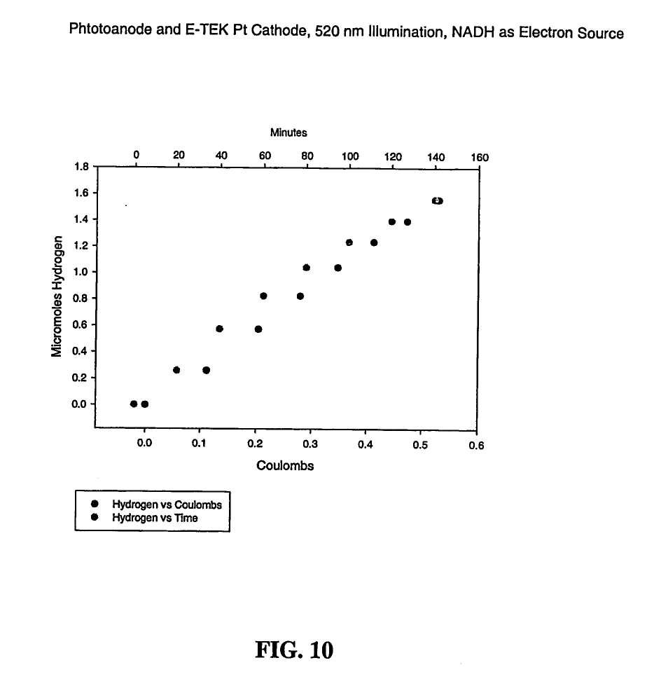

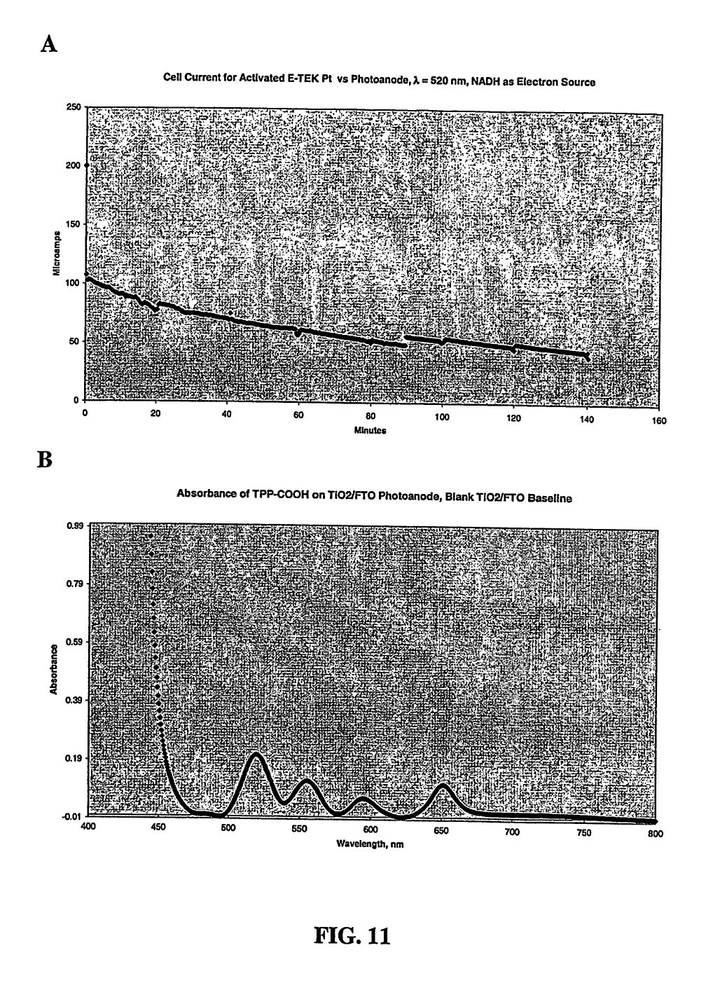

5) During this illumination process hydrogen was measured at 20 minute intervals. Each hydrogen measurement involved injecting 0.7 cc of Ar into the cathode cell, followed by withdrawal of 0.7 cc of headspace gas. The headspace gas samples were analyzed by gas chromatography (Varian 3800-CP). The results are shown in FIG. 10.

D) Quantum Yield Calculations

Photodiode current=242.5 microamps
Light Intensity (mW/cm²)=((242.5 E⁻⁶ A/0.2928 A/W)*1000)/0.419 cm²=1.98 mW/cm²
PA area in solution=2.475 cm²
Total mW incident=(1.98 mW/cm²)*(2.475 cm²)=4.90 mW
Photons per second=[(4.90 mW)*(520 nm)*10¹³]/1.987=1.28 E¹⁶ photons/sec
IPCE:

From 0 to 140 minutes (8400 seconds)
1.56 micromoles of hydrogen produced
3.12 E⁻⁶ mol electrons in H₂
1.88 E¹⁸ electrons in H₂

From 0 to 140 minutes (8400 seconds)
(1.28 E¹⁶ photons/sec)*8400 seconds=1.07 E²⁰ photons
IPCE=(1.88 E¹⁸ electrons in H₂)/(1.07 E²⁰ incident photons)=0.0176
LHE=1−(10^{-A})
Photoanode absorbance at 520 nm=0.210
LHE=1−(10^{-0.210})=0.383
Quantum Yield=IPCE/LHE=0.0176/0.383=0.0460
Quantum Yield=4.6%

### Conversion of Low Value Organic Materials in a Closed Photobiofuel Cell System

In another embodiment, this invention comprises a method for the in situ conversion of low value/energy organic material to high value/energy material in a closed system using solar energy and the photoanode system described above. The synthesis of hydrogen using electrical current from the output side of the photoanode and the fuel and coenzymes NAD or NADP and associated enzymes that make up the input components of the photoanode is as discussed above.

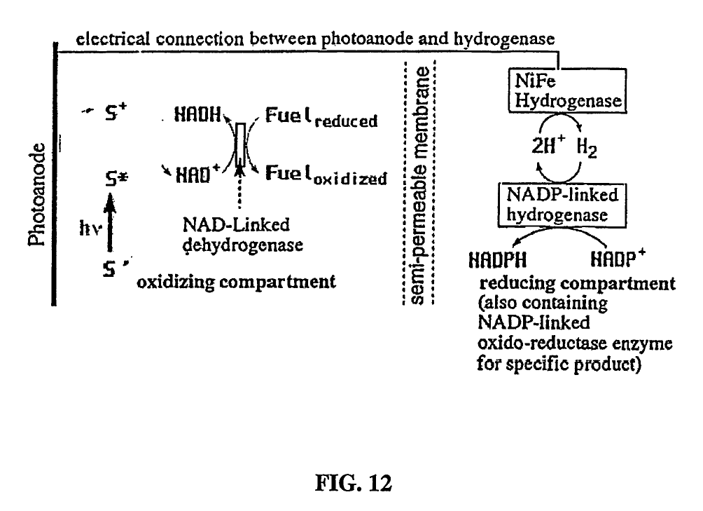

A hydrogenase enzyme that catalyzes the synthesis of hydrogen from protons and electrons is attached to the photoanode employed in the cell described above by an electrical conductor or other suitable means so that sufficient electrochemical potential is provided to produce hydrogen in a reducing compartment (See FIG. 12). This reducing compartment is linked to the photoanode compartment by a semi-permeable device, and contains, in addition to the hydrogenase enzyme responsible for hydrogen production, a second hydrogenase that utilizes the coenzyme NADP. This second, NADP-dependent hydrogenase consumes the hydrogen produced by the hydrogenase-bearing electrode, and in the process reduces NADP⁺ to NADPH and liberates a hydrogen ion. The NADPH is used to provide reducing power to other enzymes, such as oxido-reductase enzymes, which, along with their substrates, may be present in the reducing compartment, or may be in a separate compartment. These enzymes use the reducing power of NADPH to synthesize useful high value/high energy products.

The hydrogen produced may be thought of as coming from the reforming of the fuel component of the photobiofuel cell previously disclosed. The energy for the hydrogen production and therefore for the production of NADPH comes from both the light absorbed by the photoanode and any associated accessory antenna systems and the energy inherent in the fuel source of the cell previously disclosed. The method described is useful in that it converts a variety of fuel sources including carbon-containing waste materials and low energy content fuels to a valuable fuel or chemicals in the form of a reduced carbon compound in a closed system. The fuel sources include, but are not limited to, fats, hydrocarbons, carbohydrates, proteins, and any materials that are oxidized, directly or indirectly, by the photoanode. The solar energy input in this system sets the chemical potential difference in redox poise between the oxidizing and reducing compartments. This chemical potential is available for the net conversion of low value/energy fuels in the oxidizing compartment to high value/energy compounds in the reducing compartment. This may be a closed system in that only the redox states of the materials in each compartment change. For example, methanol could be reduced to methane in the reducing compartment with the concomitant oxidation of an aldehyde to an acid in the oxidizing compartment.

Hydrogenase enzymes are found in a large variety of organisms and catalyze the formation of hydrogen from protons and electrons. In order to operate, they must have a source of protons from an aqueous solution at moderate pH and electrons with sufficient reduction potential to form hydrogen (E_{1/2}=−0.42 V at pH 7).

NADP-dependent, also known as NADP-linked, hydrogenase enzymes occur in a large variety of organisms and catalyze the reduction of NADP⁺ to NADPH by molecular hydrogen.

NADP-dependent, also known as NADP-linked, oxido-reductase enzymes occur in a large variety of organisms and catalyze the reduction of specific carbon-containing compounds by NADPH in biosynthetic metabolic pathways. The exact product desired is determined by the presence of the necessary substrates and the specific NADP-dependent enzyme employed in the reducing compartment of the cell. These enzymes are shown in FIG. 12 as a NADP-linked oxido-reductase in the reducing compartment on the right.

The basis of the method is a photoelectrochemical fuel cell divided into two compartments. Fuel cells carry out oxidation and reduction chemical reactions in two half-cells, and use the resulting energy to generate electromotive force and electrical current flowing between an anode and a cathode. In this invention, electron flow from the photoanode compartment (oxidizing compartment on the left in FIG. 12) is diverted from the cathode to an enzyme, catalyzing the formation of hydrogen from protons and electrons in the reducing compartment on the right. This enzyme along with an NADP-dependent hydrogenase is located in a separate compartment, the reducing compartment, coupled to the anode compartment. The redox poise in this compartment is highly reducing when the photoanode is exposed to light and all the components necessary for it to function are present. The device is a hybrid electrochemical fuel cell consisting of:

a. An electrochemical half-cell consisting of a dye-sensitized nanoparticulate photoanode operating in an aqueous medium, which contains the reducing agent NADH or NADPH that donates electrons into the photo-oxidized dye at the photoanode, thereby being itself oxidized. This half-cell also contains appropriate reduced carbon or other fuel materials in conjunction with appropriate enzymes to provide reducing equivalents to maintain the NADH or NADPH levels. In the light the redox poise in this compartment is highly oxidizing. It is shown in FIG. 12 on the left.

b. A second electrode attached electrically to the photoanode in the oxidizing compartment and to any of the class of hydrogen producing enzymes known as hydrogenases. This hydrogenase-bearing electrode is wired to the photoanode by an electrical conductor but placed in a separate compartment, shown on the right, and coupled to the photo anode half-cell compartment via a semi-permeable device such as a membrane, frit or salt bridge. Both the wire or electrical conductor and the semi-permeable membrane/frit/salt bridge serve to couple the two compartments together. Exposure of the photo anode to actinic light sets the redox poise of this compartment highly reducing.

c. The compartment containing the hydrogenase wired to the photoanode also contains an NADP-dependent hydrogenase. This enzyme catalyzes the conversion of hydrogen to protons with the concomitant reduction of NADP+ to NADPH according to the equation NADP⁺+H₂+NADPH+H⁺. The production of NADPH at highly reducing potentials enables the synthesis of high value/energy compounds.

d. The compounds to be synthesized are determined by the presence of an additional specific enzyme, an NADP-linked oxido-reductase, or enzymes that catalyze the formation of the product of interest by utilizing the reduction potential provided by the NADPH and other substrates that may be necessary.

e. A suitable light source.

Details of Cell Function.

Photo anode. One component of the photoanode (left side of FIG. 12) is a conductive surface, which may be transparent to light. Indium tin oxide coated glass or indium tin oxide coated fused silica are examples of a transparent conductive material. The surface of this material is covered with a layer of wide band gap semiconductor nanoparticles. Examples of suitable materials are tin dioxide and titanium dioxide. Onto the surface of the nanoparticles is deposited a layer of light-absorbing sensitizer dye (S in FIG. 12). This material absorbs light in spectral regions where the excitation source with which the cell will be used has a significant photon flux. The energies of some of its lowest-lying excited states (singlet, triplet) and its first oxidation potential are such that the singlet or triplet state is energetically capable of injection of an electron into the nanoparticulate surface to generate S⁺. This event leads to formation of the oxidized sensitizer, S⁺, and mobile electrons which migrate through the nanoparticulate layer to the conductive surface, and hence into the external electrical circuit. The sensitizer S bears functionality that allows it to bind to the surface of the nanoparticles in a structural arrangement that renders the excited state of S kinetically competent to inject an electron into the nanoparticle layer with suitable efficiency.

Redox Couple. The redox couple consists of NADH/NAH⁺ or NADPH/NADP⁺, depending upon which species is active towards the fuel material to be oxidized. A mixture may be used with appropriate enzymes to accommodate a variety of fuel sources. As the cell operates upon illumination of the photoanode, the NADH or NADPH present is converted into NAH+ or NADP⁺. In this process, two electrons are removed from NADH or NADPH by the oxidized sensitizer S⁺, regenerating S, and electrons pass through the external circuit. The fuel materials in concert with appropriate enzymes regenerate the NADH or NADPH and become oxidized themselves. Thus, S is recycled as a photocatalyst, and NADH or NADPH are recycled, and the electrons flowing through the circuit are used by the hydrogenase to produce hydrogen from protons.

Electrolyte. The half-cell electrolyte is aqueous in nature, providing an environment in which the photoanode, redox couple, and hydrogenases are capable of functioning as described above. The electrolyte contains any necessary buffers, salts or other substances necessary to ensure stable operation of the cell. The electrolyte may be different in the two compartments and optimized for the performance of the redox enzymes in each compartment.

Hydrogenase Enzyme. Electrons flowing from the photoanode through an appropriate electrical conduit to the hydrogenase (e.g., NiFe Hydrogenase in FIG. 12) will have sufficient reducing potential to drive the synthesis of hydrogen from protons and electrons according to the chemical equation: 2H⁺+2^{-e}→H₂. The hydrogenase enzymes include, but are not limited to, the NiFe hydrogenases from organisms such as Escherichia coli, Nostoc muscorum, Rhodospirillum rubrum, Rhodobacter capsulatus, Chromatium vinosum and others. Fe-only hydrogenases from organisms such as Clostridiium acetobutylicum and others may also be used. The hydrogenase electrode connection could be as described by Reshad, et al. (1999) Biochemistry 38: 8992-8999 or by other suitable means involving modified electrodes and molecular-level electrical contact techniques.

NADP-linked hydrogenase enzyme. Electrons are carried to this catalyst by the hydrogen produced as described above and are used to reduce NADP⁺ to NADPH. The necessary reduction potential is provided by the photoanode, via the hydrogen, and is sufficient to poise the NADP⁺/NADPH redox couple reducing.

NADPH-linked oxido-reductase enzyme. A wide variety of these enzymes are found in the reductive biosynthetic pathways of living organisms. The specific one chosen would depend on the high value/energy reduced carbon-containing compound desired.

Configuration. This invention could be used as shown in FIG. 12 with the photoanode and the fuel source previously disclosed or wired in parallel with an oxygen reducing cathode in the reducing compartment in the solar photobiofuel cell, as described above. This is possible because certain of the hydrogenase enzymes are self-regulating in that, in the presence of oxygen, they are inhibited. Under oxygenated conditions, the device acts as a light-energy to electrical-energy converting device with current flow from the photoanode to the cathode. In the absence of oxygen or when the concentration of oxygen is low, the current flow to the cathode (where oxygen is consumed) is slow, oxygen inhibition of the hydrogenase enzyme is reduced, and hydrogen synthesis and NADPH production are accelerated. Functioning in this mode the hydrogen and NADPH and reduced fuel compounds in the reducing compartment are energy storage media, and the system could incorporate solid state storage media for appropriate energy densities for the hydrogen.

In the dark or under heavy current demand, the hydrogen and reduced fuel compounds produced and stored would be oxidized by the hydrogenase and NADPH-linked oxido-reductase, respectively, and would supply electrons to the anode and thereby replace the photocatalytic production of electrons by the photobiofuel cell. Alternatively, this process could be used in addition to illumination of the photoanode and consumption of a fuel, thus augmenting the output of the photobiofuel cell. Operation in this mode would require an oxygen tolerant hydrogenase or a means of excluding oxygen from the hydrogenase enzyme, and in this sense is an alternative to the self-regulated operation discussed above.

While this invention is described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. In particular, it is to be understood that this invention is not limited to the particular methodology, protocols, cell lines, animal species or genera, constructs, and reagents described as such may vary, as will be appreciated by one of skill in the art.

What is claimed is:

1. A method for producing hydrogen, comprising the steps of:
providing a photobiofuel cell comprising:
a. an electrochemical half-cell comprising a dye-sensitized photoanode operating in an aqueous medium, said medium comprising NADH, a fuel, and an enzyme selected to provide reducing equivalents to maintain NADH levels;
b. an electrode, the electrode electrically coupled to a catalyst and connected to the photoanode by an electrical conductor; and
c. a light source; and
illuminating the photoanode with light to thereby produce hydrogen.

2. The method of claim 1, wherein the photoanode comprises indium tin oxide coated glass.

3. The method of claim 2, wherein the photoanode further comprises a layer of semiconductor nanoparticles.

4. The method of claim 3, wherein the nanoparticles comprise tin oxide.

5. The method of claim 3, wherein the nanoparticles comprise titanium dioxide.

6. The method of claim 1, wherein the photoanode comprises indium tin oxide coated fused silica.

7. The method of claim 6, wherein the photoanode further comprises a layer of semiconductor nanoparticles.

8. The method of claim 7, wherein the nanoparticles comprise tin oxide.

9. The method of claim 7, wherein the nanoparticles comprise titanium dioxide.

10. The method of claim 1, wherein the catalyst is an hydrogenase.

11. The method of claim 10, wherein the hydrogenase is NiFe hydrogenase.

12. The method of claim 1, wherein the catalyst comprises platinum.

13. The method of claim 12, wherein the catalyst is E-TEK Pt/C.

14. The method of claim 1, wherein the catalyst-coupled electrode is contained within the photoanode half-cell.

15. The method of claim 1, wherein the catalyst-coupled electrode is coupled to the photoanode half-cell via a semi-permeable device.

16. The method of claim 15, where int the semi-permeable device is selected from the group consisting of a membrane, a frit and a salt bridge.

17. The method of claim 1, wherein the fuel is reduced carbon.

18. A method for producing hydrogen, comprising the steps of:
providing a photobiofuel cell comprising:
a. an electrochemical half-cell comprising a dye-sensitized photoanode operating in an aqueous medium, said medium comprising NADPH, a fuel, and an enzyme selected to provide reducing equivalents to maintain NADPH levels;
b. an electrode, the electrode electrically coupled to a catalyst and connected to the photoanode by an electrical conductor; and
c. a light source; and
illuminating the photoanode with light to thereby produce hydrogen.

19. The method of claim 18, wherein the photoanode comprises indium tin oxide coated glass.

20. The method of claim 19, wherein the photoanode further comprises a layer of semiconductor nanoparticles.

21. The method of claim 20, wherein the nanoparticles comprise tin oxide.

22. The method of claim 20, wherein the nanoparticles comprise titanium dioxide.

23. The method of claim 18, wherein the photoanode comprises indium tin oxide coated fused silica.

24. The method of claim 23, wherein the photoanode further comprises a layer of semiconductor nanoparticles.

25. The method of claim 24, wherein the nanoparticles comprise tin oxide.

26. The method of claim 24, wherein the nanoparticles comprise titanium dioxide.

27. The method of claim 18, wherein the catalyst is an hydrogenase.

28. The method of claim 27, wherein the hydrogenase is NiFe hydrogenase.

29. The method of claim 27, wherein the catalyst comprises platinum.

30. The method of claim 29, wherein the catalyst is E-TEK Pt/C.

31. The method of claim 18, wherein the catalyst-coupled electrode is contained within the photoanode half-cell.

32. The method of claim 18, wherein the catalyst-coupled electrode is coupled to the photoanode half-cell via a semi-permeable device.

33. The method of claim 32, where int the semi-permeable device is selected from the group consisting of a membrane, a frit and a salt bridge.

34. The method of claim 18, wherein the fuel is reduced carbon.

35. A method for converting low energy organic material to high energy material, comprising the steps of:
providing an electrochemical fuel cell comprising:
a. an electrochemical half-cell comprising a dye-sensitized nanoparticulate photoanode operating in an aqueous medium, said medium comprising NADH, a low energy fuel material, and an enzyme selected to provide reducing equivalents to maintain NADH levels;
b. a compartment comprising an electrode, an NADP-dependent hydrogenase, a catalyst and an NADP-dependent oxido-reductase enzyme, the electrode electrically coupled to the catalyst and connected to the photoanode by an electrical conductor, wherein the compartment is coupled to the electrochemical half cell by a semi-permeable device; and
c. a light source; and
illuminating the photoanode with light to thereby convert the low energy fuel material to high energy fuel material.

36. The method of claim 35, wherein the catalyst comprises an hydrogenase.

37. The method of claim 36, wherein the hydrogenase is NiFe hydrogenase.

38. The method of claim 35, wherein the catalyst comprises platinum.

39. The method of claim 38, wherein the catalyst is E-TEK Pt/C.

40. A method for converting low energy organic material to high energy material, comprising the steps of:
providing an electrochemical fuel cell comprising:
a. an electrochemical half-cell comprising a dye-sensitized nanoparticulate photoanode operating in an aqueous medium, said medium comprising NADPH, a low energy fuel material, and an enzyme selected to provide reducing equivalents to maintain NADPH levels;
b. a compartment comprising an electrode, an NADP-dependent hydrogenase, a catalyst and an NADP-dependent oxido-reductase enzyme, the electrode electrically coupled to the catalyst and connected to the photoanode by an electrical conductor, wherein the compartment is coupled to the electrochemical half cell by a semi-permeable device; and
c. a light source; and
illuminating the photoanode with light to thereby convert the low energy fuel material to high energy fuel material.

41. The method of claim 40, wherein the catalyst comprises an hydrogenase.

42. The method of claim 41, wherein the hydrogenase is NiFe hydrogenase.

43. The method of claim 40, wherein the catalyst comprises platinum.

44. The method of claim 43, wherein the catalyst is E-TEK Pt/C.

45. The method of claim 1, wherein the photoanode comprises fluorine tin oxide coated fused silica.

46. The method of claim 18, wherein the photoanode comprises fluorine tin oxide coated glass.

47. The method of claim 18, wherein the photoanode comprises fluorine tin oxide coated fused silica.
